(19) United States
(12) Patent Application Publication
(10) Pub. No.: US 2026/0256401 A1
(43) Pub. Date: Sep. 3, 2026

(54) MOTOR-RELATED ELECTROENCEPHALOGRAM (EEG) SOURCE LOCALIZATION METHOD AND SYSTEM BASED ON MINIMUM NORM ESTIMATION-GENERATIVE ADVERSARIAL NETWORK (MNE-GAN)

(71) Applicant: SOUTHEAST UNIVERSITY, Jiangsu (CN)

(72) Inventors: Baoguo XU, Jiangsu (CN); Yuheng ZHANG, Jiangsu (CN); Yongqiang SU, Jiangsu (CN); Aiguo SONG, Jiangsu (CN)

(73) Assignee: SOUTHEAST UNIVERSITY, Jiangsu (CN)

(21) Appl. No.: 19/653,055

(22) Filed: Apr. 20, 2026

Related U.S. Application Data
(63) Continuation of application No. PCT/CN2025/123158, filed on Sep. 23, 2025.

(30) Foreign Application Priority Data
Jun. 5, 2025 (CN) 202510744870.8

(51) Int. Cl. A61B 5/318

(52) U.S. Cl. CPC A61B 5/318

(57) ABSTRACT

The present disclosure provides a motor-related electroencephalogram (EEG) source localization method and system based on minimum norm estimation-generative adversarial network (MNE-GAN). The method includes the following steps: synthesizing virtual multi-channel EEG data; constructing an EEG source data generation network based on minimum norm estimation (MNE); constructing an EEG source data discriminator; preprocessing virtual EEG data; training an MNE-GAN model based on the virtual EEG data; and performing source localization of real EEG data using the trained model. The present disclosure provides a novel EEG source localization method based on an MNE-based generative adversarial network. By introducing a minimum norm constraint, the generated EEG source data are constrained to satisfy physical prior knowledge, which is beneficial for non-invasive neurophysiological mechanism studies and for improving EEG decoding accuracy.

### CROSS-REFERENCE TO RELATED APPLICATION

This application is a continuation of international application of PCT application serial no. PCT/CN2025/123158 filed on Sep. 23, 2025, which claims the priority benefit of China application no. 202510744870.8 filed on Jun. 5, 2025. The entirety of each of the above-mentioned patent applications is hereby incorporated by reference herein and made a part of this specification.

### TECHNICAL FIELD

The present disclosure relates to the technical field of Electroencephalogram (EEG) source localization, and in particular to a motor-related EEG source localization method and system based on minimum norm estimation-generative adversarial network (MNE-GAN).

### BACKGROUND

Electroencephalogram (EEG) is a technique that uses electrodes placed along the scalp to capture weak electrical activity of neuronal populations in the cerebral cortex. Since the German scientist Hans Berger first recorded such signals from the human scalp, its millisecond-level temporal resolution has made it a pivotal tool for tracking dynamic changes in brain function (including analysis of cognitive processes, recognition of emotional states, and decoding of motor intentions) and for clinical diagnosis (such as localization of epileptic foci and analysis of sleep stages). However, due to the attenuation effect of skull conductivity and the influence of volume conductor effect, the spatial resolution of scalp EEG signals is significantly limited, making it difficult to achieve precise spatial localization of intracranial neural activity.

To address the problem of low spatial resolution of EEG signals, EEG source localization techniques have been gradually developed. By establishing a head volume conductor model and solving the inverse problem, these techniques reconstruct the three-dimensional spatial distribution characteristics of neural electrical activity within the brain based on the scalp potential distribution. However, the solution of this problem is essentially a mathematical “inverse problem,” which exhibits non-uniqueness and is highly dependent on the accuracy of the head volume conductor model. Existing source localization methods have significant limitations. Although traditional algorithms based on minimum norm estimation (MNE) and standardized low-resolution electromagnetic tomography (sLORETA) constrained the solution space by introducing physical prior constraints, they are prone to introducing systematic localization errors due to excessive simplification of head geometric models. Furthermore, traditional algorithms lack robustness and are sensitive to measurement noise such as electromyographic artifacts and electrode impedance variations, which easily leads to distortion of source localization results. In addition, existing methods are mostly based on the assumption of static neural activity, making it difficult to effectively capture the spatiotemporal evolutionary characteristics of dynamic neural processes.

Deep learning has demonstrated significant advantages in EEG source localization, overcoming the limitations of traditional methods that rely on simplified models. It automatically learns the complex relationships between EEG signals and neural activity in a data-driven manner. Its dynamic modeling capability enables real-time tracking of transient neural activity, and effectively improves localization reliability in combination with noise-resistant design and multimodal fusion techniques. End-to-end architectures also greatly improve computational efficiency, enabling real-time imaging and promoting applications in precision medicine. These technical characteristics make deep learning an important research direction for optimizing the performance of EEG source localization.

### SUMMARY

In view of the deficiencies in the prior art, the present disclosure provides a motor-related EEG source localization method and system based on MNE-GAN. By introducing a minimum norm constraint into a deep neural network for EEG source localization, a new possibility is provided for in-depth analysis of EEG signals.

In order to achieve the above objectives, the present disclosure adopts the following technical solutions:

In a first aspect, the present disclosure provides a motor-related EEG source localization method based on MNE-GAN, including the following steps:
(1) generating virtual EEG source data in a motor state according to the Brodmann areas, computing an EEG forward propagation process based on a realistic head model to obtain a lead field matrix, mapping the virtual EEG source data to scalp electrode positions, and introducing electromyographic (EMG), electrocardiogram and electrooculographic (EOG) noises after the mapping process to generate EEG signals that simulate a real acquisition environment;
(2) preprocessing the EEG signals, including re-referencing, band-pass filtering, downsampling, artifact removal, and epoching, and extracting effective EEG features;
(3) constructing a generative adversarial network model based on minimum norm estimation (MNE-GAN model); where in the MNE-GAN model, a generator is configured to use the preprocessed virtual EEG data as a training set, learn spatial-temporal features of EEG signals through multi-layer convolution operations, and output high-resolution source-space averaged EEG signals in a block-wise manner according to the Brodmann areas, thereby establishing a mapping model from EEG signals to brain source distributions; and a discriminator is configured to use the source-space averaged EEG signals generated by the generator as fake samples and the EEG signals simulated in the step (1) as real samples for adversarial training, use binary cross-entropy loss as a loss function, compute a generator loss based on the discrimination results, and introduce a minimum norm inverse solution into the generator loss;
(4) training the MNE-GAN model using a staged training strategy: first, training the discriminator using noise signals, alternately training the generator and the discriminator until the model converges, and finally introducing the minimum norm inverse solution as a supervisory term for joint optimization; and
(5) preprocessing real EEG data according to the method as described in the step (2), inputting the preprocessed data into the trained MNE-GAN model, outputting signals corresponding to specific regions of the cerebral cortex, and performing localization according to intensities of the output signals.

In a second aspect, the present disclosure provides a motor-related EEG source localization system based on MNE-GAN, including:
generating virtual EEG source data: generating virtual EEG source data in a motor state according to Brodmann areas, computing an EEG forward propagation process based on a realistic head model to obtain a lead field matrix, mapping the virtual EEG source data to scalp electrode positions, introducing electromyographic (EMG), electrocardiogramd electrooculographic (EOG) noises after the mapping process to generate EEG signals that simulate a real acquisition environment, and saving the EEG source data and the simulated EEG data;
an EEG signal preprocessing module, configured to preprocess the EEG signals, including re-referencing, band-pass filtering, downsampling, artifact removal, and epoching, and extracting effective EEG features;
an MNE-GAN model construction module, configured to construct a generative adversarial network model based on minimum norm estimation (MNE-GAN model); in the MNE-GAN model, the generator uses the preprocessed virtual EEG data as a training set, learns spatial-temporal features of EEG signals through multi-layer convolution operations, and outputs high-resolution source-space averaged EEG signals in a block-wise manner according to the Brodmann areas, thereby establishing a mapping model from EEG signals to brain source distributions; and the discriminator uses the source-space averaged EEG signals generated by the generator as fake samples and the EEG signals simulated by the virtual EEG source data generation module as real samples for adversarial training, uses binary cross-entropy loss as a loss function, computes generator loss based on the discrimination results, and introduces a minimum norm inverse solution into the generator loss;
a model training module, configured to train the MNE-GAN model using a staged training strategy: first the discriminator is trained using noise signals, the generator and the discriminator are then alternately trained until the model converges, and finally the minimum norm inverse solution is introduced as a supervisory term for joint optimization; and a source localization module, configured to preprocess real EEG data according to the method of the EEG signal preprocessing module, input the preprocessed data into the trained MNE-GAN model, output signals corresponding to specific regions of the cerebral cortex, and perform localization according to intensities of the output signals.

In a third aspect, the present disclosure provides a computer device, including: one or more processors; a memory; and one or more programs, where the one or more programs are stored in the memory and are configured to be executed by the one or more processors, and when the programs are executed by the processors, the steps of the motor-related EEG source localization method based on MNE-GAN as described in the first aspect are implemented.

In a fourth aspect, the present disclosure provides a computer-readable storage medium having a computer program stored thereon, where when the computer program is executed by a processor, the steps of the motor-related EEG source localization method based on MNE-GAN as described in the first aspect are implemented.

### BRIEF DESCRIPTION OF THE DRAWINGS

FIG. 1 is a main flowchart of a method according to the present disclosure.

FIG. 2 is a schematic diagram of motor-related brain regions.

FIG. 3 is a schematic diagram of constructing a generator model.

FIG. 4 is a schematic diagram of constructing a discriminator model.

### DETAILED DESCRIPTIONS OF THE EMBODIMENTS

The technical solutions of embodiments of the present disclosure will be described below clearly and comprehensively in conjunction with accompanying drawings of the present disclosure.

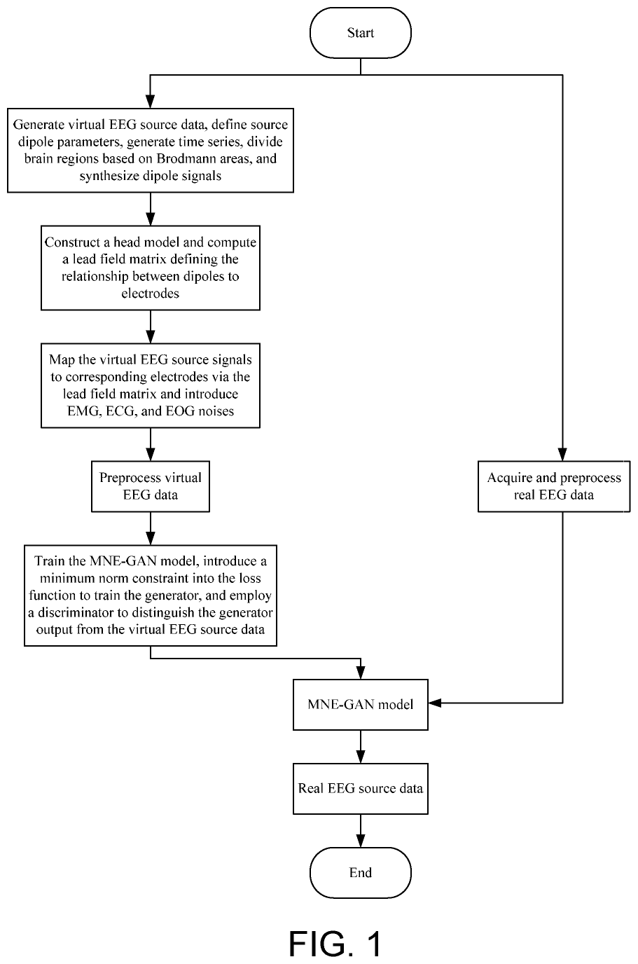

The embodiments of the present disclosure provide a motor-related EEG source localization method based on MNE-GAN. Referring to FIG. 1, the method includes the following steps:
(1) generating virtual EEG source data: generating virtual EEG source data by adopting an MNE-Python Toolkit according to the Brodmann areas, computing an EEG forward propagation process based on a realistic head model to obtain a lead field matrix, mapping the virtual EEG source data to scalp electrode positions, introducing electromyographic (EMG), electrocardiogram and electrooculographic (EOG) noises after the mapping process to generate EEG signals R(x) that simulate a real acquisition environment, and saving the EEG source data and the simulated EEG data.

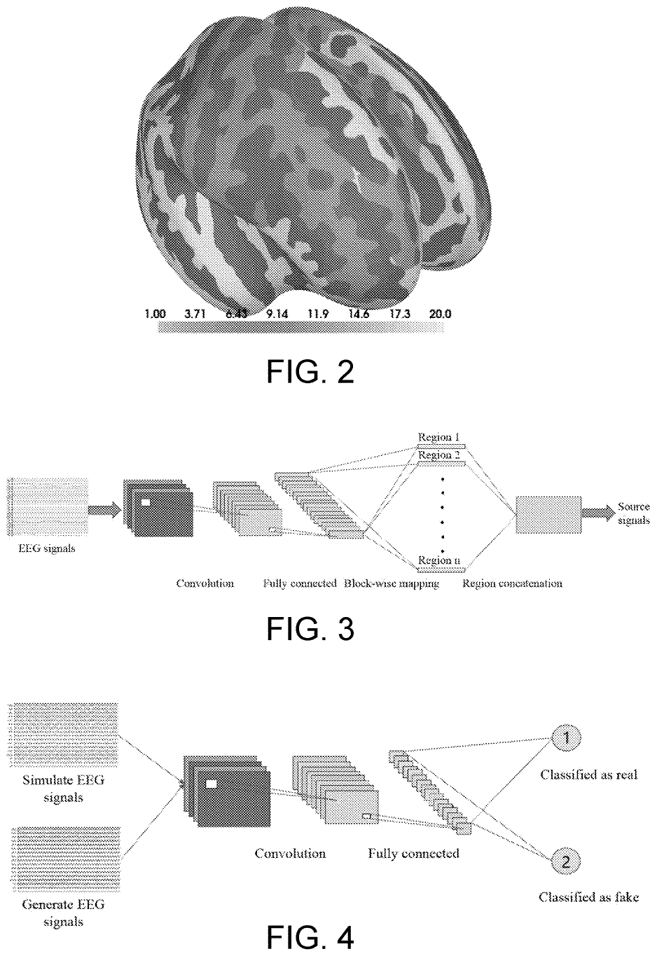

Specifically, the step (1) includes:
(1.1) a schematic diagram of motor-related brain regions is shown in FIG. 2. The highlighted areas in the figure represent 26 motor-related brain regions selected according to the Brodmann areas, including a primary somatomotor cortex (G_precentral), supplementary/premotor motor area (S_precentral), supplementary motor area (G_and_S_paracentral), cortical-cortical motor integration area (G_and_S_subcentral), and other limbic motor-related regions. Among the upper-limb motor-related regions, the primary somatomotor cortex and the supplementary/premotor motor areas are defined as the primary activation regions; the postcentral gyrus (G_postcentral), supplementary motor area (G_and_S_paracentral), central sulcus (S_central), and cortical-cortical motor integration area (G_and_S_subcentral) are defined as synergistic regions; and the remaining motor-related regions are defined as background regions;
(1.2) source dipoles of the selected 26 motor-related brain regions are initialized using blank signals with a duration of 5 s and a sampling frequency of 256 Hz. In the selected primary activation regions, during 0-1 s, a μ rhythm is simulated using sinusoidal waves with random frequencies of 8-13 Hz and random amplitudes of 5-10 μV, and a β rhythm is simulated using sinusoidal waves with random frequencies of 13-30 Hz and random amplitudes of 10-20 μV. During 1-3 s, the amplitudes of the u rhythm and β rhythm are gradually reduced to 30% of their original amplitudes. During 3-5 s, the μ rhythm remains unchanged, while the amplitude of the β rhythm is gradually increased back to its original amplitude. In the synergistic regions, EEG activity during movement is simulated using 50% of the amplitude of the primary activation regions. In the background regions, background noise and other low-frequency activities are simulated using random noise of 0.1-2 μV. Corresponding signals are assigned to the dipoles in different regions to obtain an overall simulated signal; and
(1.3) according to the forward boundary element method (BEM) head model, a conductivity value of the three-layer model is set (scalp: 0.3 S/m; skull: 0.006 S/m; and brain: 0.3 S/m); a forward solution of EEG signals in the motor-related regions is computed, and the EEG signals in a source space are mapped onto the electrodes; and during this process, ECG, EMG, and EOG noises, as well as a random noise, are introduced to simulate EEG signals acquired under realistic conditions.
(2) Preprocessing the EEG signals generated in the step (1), including re-referencing, band-pass filtering, downsampling, artifact removal, and epoching, and extracting effective EEG features.

Specifically, the step (2) includes:
(2.1) band-pass filtering is performed on the data in a range of 0.3 Hz-45 Hz, outliers are removed, and bad channels are repaired using a linear interpolation method using data from channels adjacent to bad channels;
(2.2) EMG, ECG, and EOG interferences in the EEG signals are removed using an independent component analysis (ICA) algorithm; and
(2.3) the processed signals are segmented, and baseline correction and common average referencing are performed.
(3) Constructing a convolutional neural network (CNN) based on minimum norm estimation (MNE) as a generator. Referring to FIG. 3, the preprocessed virtual EEG data from the step (2) are used as a training set. Through multi-layer convolution operations, spatial-temporal features of EEG signals are learned. High-resolution source-space averaged EEG signals are output in a block-wise manner according to the Brodmann areas, thereby establishing a mapping model from EEG signals to EEG source distributions.

Specifically, the step (3) includes:
(3.1) The generator adopts a convolutional neural network (CNN) combined with fully connected layers to extract EEG signal features, the preprocessed EEG signal X∈R^{c×t}, with c electrode channels and time length t, is taken as input and fed into a convolutional layer for feature extraction, and dimensionality reduction is performed through a pooling layer; and the input feature of a k^{th} layer is h^{k-1}, and the convolution operation of this layer may be expressed as: h^{k}=ƒ(Conv(h^{k-1},ω^{k},b^{k})), where ƒ(⋅) denotes a ReLU activation function, w denotes weight parameters, and b denotes bias. One pooling operation is performed after every two convolution operations to reduce the overall computational load. A total of five convolution operations are conducted to finally obtain the EEG encoded data s;
(3.2) the fully connected layers are used to decode the data s encoded by the convolutional layers, and two fully connected layers are used for computation; and the process may be expressed as: h^{m}=ƒ(ω^{m}*h^{m-1}+b^{m}), where ƒ(⋅) denotes a ReLU activation function, ω denotes weight parameters, and b denotes bias. A linear activation function is used to finally obtain the EEG features;
(3.3) the obtained EEG features are mapped in a block-wise manner; the motor-related regions are selected according to the Brodmann areas, and each block is separately mapped using a single-layer convolution. Assuming that each block has r signal sources, a single source-averaged EEG signal A(x)∈R^{1*t} corresponding to an average signal of r signal sources in the source space is obtained by mapping, and all blocks are concatenated to output the overall source-averaged EEG signal G(x).

In the context of the present disclosure, the “source-space averaged EEG signal” and the “source-averaged EEG signal” are used interchangeably, both referring to EEG signals obtained through region-wise averaging. The source-averaged EEG signal in the step (1) refers to the signal obtained by averaging the dipole signals within each region, while the signal in the step (3) refers to the source-averaged EEG signal directly generated by the generator. The EEG source signal refers to the initial signal containing a plurality of dipole signals in each region without averaging.
(4) Constructing a discriminator model. Referring to FIG. 4, the source-averaged EEG signals generated in the step (3) are labeled as fake samples (label 0), while the EEG source signals generated in the step (1) are averaged by dipoles according to the areas to obtain source-averaged EEG signals, which are labeled as real samples (label 1). Both are input into the discriminator for adversarial training. Parameters of the discriminator are updated through a binary cross-entropy loss, and the generator loss is calculated based on the discrimination results. In the generator loss, the conventional minimum norm estimation (MNE) is introduced as a supervisory signal to enhance the physical plausibility of the source-space distribution.

Specifically, the step (4) includes:
(4.1) Assuming that a signal input to the discriminator is S(x); and after passing through three convolutional layers and two fully connected layers, the signal is then output through a Sigmoid activation function to obtain a probability D(S(x)) that an input sample is a real sample;
(4.2) a loss function of the discriminator is calculated using binary cross-entropy, a loss for the real sample is −log(D(R(x))), where a probability D(R(x)) that a real sample is correctly identified should be as close to 1 as possible; a loss for a generated sample is −log(1−D(G(x))), where a probability D(G(x)) that the generated sample is identified as a real sample should be as close to 0 as possible, and a final loss of the discriminator is an average of the real sample loss and the generated sample loss:

$${Loss}_{Discriminator} = - \frac{1}{N} {\sum }_{i = 1}^{N} - log ( D ( R ( {x}_{i} ) ) ) - log ( 1 - D ( G ( {x}_{i} ) ) )$$

(4.3) an adversarial loss may be calculated based on the probability D(S(x)) output by the discriminator that an input sample is a real sample:

$${Loss}_{GAN} = - \frac{1}{N} {\sum }_{i = 1}^{N} - log ( D ( G ( {x}_{i} ) ) )$$

To avoid significant fluctuations in the early stage of training, an initial loss function of the generator is only the adversarial loss. After the generated results become stable, a minimum norm constraint loss function (MNE) is introduced:

$${Loss}_{mne} = \frac{1}{N} {\sum }_{i = 1}^{N} {‖ G ( {x}_{i} ) - {G}_{mne}^{i} ‖}_{2}^{2}$$

where G(xᵢ) denotes an output of the generator, and

$${G}_{mne}^{i}$$

denotes a minimum norm inverse solution corresponding to data xᵢ. An inverse solution MNE(x) is computed using a Python Toolkit. According to the Brodmann areas, source signals of the motor-related regions are selected to serve as final results, and regional signals are averaged to obtain a minimum norm inverse solution reference

$${G}_{mne}^{i} .$$

After the minimum norm constraint loss is introduced, an explicit guidance is provided for the generated EEG source data, further enhancing the reliability of the generated EEG data. The final loss function is obtained by weighting the adversarial loss and the minimum norm constraint loss:

$${Loss}_{Ge nerator} = {Loss}_{G A N} + {γ}_{mne} * {Loss}_{mne}$$

γₘₙₑ denotes a weighting coefficient of the minimum norm loss and is used to balance the influence of various loss terms.
(5) Training the MNE-GAN model using a staged training strategy: first alternately training the discriminator and the generator until the model reaches preliminary convergence, and then introducing physical constraints and supervisory terms for joint optimization to ensure model stability and localization accuracy.

Specifically, the step (5) includes:
(5.1) pretraining the GAN network by fixing parameters of the generator, and iteratively updating parameters of the discriminator by using noise as fake samples and virtual source-averaged EEG signals as real samples;
(5.2) performing adversarial training using the pretrained discriminator and the well-designed generator, and alternately iterating the parameters until convergence;
(5.3) after the model reaches preliminary convergence, introducing Lossₘₙₑ and γₘₙₑ for joint training until LOSS Generator decreases and converges.
(6) Performing preprocessing on real EEG data, including re-referencing, band-pass filtering, downsampling, artifact removal, and epoching, and extracting effective EEG features.
(7) Inputting the real EEG data processed in the step (6) into the trained MNE-GAN model for source localization. The model outputs the acquired EEG signals as signals corresponding to specific regions of the cerebral cortex, i.e., source signals, and localization is then performed according to signal intensity. Further, the source signals derived from the model and those obtained by the MNE method are simultaneously used for an action classification task to compare the classification accuracy of the two methods. The classification accuracy is used to evaluate the generation performance.

Based on the same technical concept as the method embodiment, another embodiment of the present disclosure provides a motor-related EEG source localization system based on MNE-GAN, including:
a virtual EEG source data generation module, configured to generate virtual EEG source data in a motor state according to the Brodmann areas, compute an EEG forward propagation process based on a realistic head model to obtain a lead field matrix, map the virtual EEG source data to scalp electrode positions, and introduce electromyographic (EMG), electrocardiogram (ECG), and electrooculographic (EOG) noises after the mapping process to generate EEG signals R(x) that simulate a real acquisition environment;
an EEG signal preprocessing module, configured to preprocess the EEG signals, including re-referencing, band-pass filtering, downsampling, artifact removal, and epoching, and extracting effective EEG features.
an MNE-GAN model construction module, configured to construct a generative adversarial network model based on minimum norm estimation (MNE-GAN model); in the MNE-GAN model, the generator uses the preprocessed virtual EEG data as a training set, learns spatial-temporal features of EEG signals through multi-layer convolution operations, and outputs high-resolution source-space averaged EEG signals in a block-wise manner according to the Brodmann areas, thereby establishing a mapping model from EEG signals to brain source distributions; and the discriminator uses the source-space averaged EEG signals generated by the generator as fake samples and the EEG signals simulated by the virtual EEG source data generation module as real samples for adversarial training, uses binary cross-entropy loss as a loss function, computes generator loss based on the discrimination results, and introduces a minimum norm inverse solution into the generator loss;
a model training module, configured to train the MNE-GAN model using a staged training strategy: first the discriminator is trained using noise signals, the generator and the discriminator are then alternately trained until the model converges, and finally the minimum norm inverse solution is introduced as a supervisory term for joint optimization; and
a source localization module, configured to preprocess real EEG data according to the method of the EEG signal preprocessing module, input the preprocessed data into the trained MNE-GAN model, output signals corresponding to specific regions of the cerebral cortex, and perform localization according to intensities of the output signals.

It should be understood that the motor-related EEG source localization system based on MNE-GAN in this embodiment can implement all the technical solutions in the above method embodiments. The functions of it various functional modules can be implemented according to the methods described in the above method embodiments, and the specific implementation processes may refer to the relevant descriptions in the above embodiments and will not be repeated herein.

Another embodiment of the present disclosure provides a computer device, including: one or more processors; a memory; and one or more programs, where the one or more programs are stored in the memory and are configured to be executed by the one or more processors, and when the programs are executed by the processors, the programs implement the steps of the motor-related EEG source localization method based on MNE-GAN as described above.

Another embodiment of the present disclosure provides a computer-readable storage medium having a computer program stored thereon, where when the computer program is executed by a processor, the steps of the motor-related EEG source localization method based on MNE-GAN as described above are implemented.

Those skilled in the art should understand that the embodiments of the present disclosure may be provided as a method, an apparatus (system), a computer device, or a computer program product. Therefore, the present disclosure may employ full hardware examples, full software examples, or software and hardware combined examples. Moreover, the present disclosure may take the form of a computer program product implemented on one or more computer usable storage media (including, but not limited to, disk memories, CD-ROM, optical memories, and the like) containing computer usable program codes.

The present disclosure is described with reference to flowcharts of the method according to embodiments of the present disclosure. It should be understood that each process in the flowcharts and combinations of processes in the flowcharts may be implemented by computer program instructions. These computer program instructions may be provided to a processor of a general-purpose computer, a special-purpose computer, an embedded processor, or other programmable data processing apparatus to produce a machine, such that the instructions executed by the processor of the computer or other programmable data processing apparatus create means for implementing the functions specified in one or more processes of the flowcharts.

These computer program instructions may also be stored in a computer-readable memory capable of directing a computer or other programmable data processing apparatus to function in a particular manner, such that the instructions stored in the computer-readable memory produce an article of manufacture including instruction means for implementing the functions specified in one or more processes of the flowcharts.

These computer program instructions may also be loaded onto a computer or other programmable data processing apparatus to cause a series of operational steps to be performed on the computer or other programmable apparatus to produce a computer-implemented process, such that the instructions executed on the computer or other programmable apparatus provide steps for implementing the functions specified in one or more processes of the flowcharts.

Beneficial effects:
(1) The present disclosure constructs a model for solving the inverse problem of EEG signals by using a generative adversarial network (GAN). Compared with conventional EEG source localization methods, the method provided by the present disclosure overcomes the limitations of conventional methods that rely on simplified models, thereby improving localization accuracy and stability, and providing a new strategy for EEG source localization.
(2) Through the adversarial training mechanism, the present disclosure can identify and suppress the effects of noise, improving the robustness of the inverse solution. In combination with the minimum norm constraint, the method can effectively reduce the interference of noise on the inverse solution while maintaining solution accuracy.
(3) The present disclosure emphasizes the importance of simulating EEG signals. By integrating brain activity mechanisms with Brodmann areas, the present disclosure designs a method for generating simulated EEG signals, enabling a more realistic simulation of actual brain activity.
(4) The present disclosure uses the minimum norm inverse solution as a constraint to limit the range of the inverse solution, thereby preventing overfitting and enhancing the interpretability of the model.

What is claimed is:

1. A motor-related electroencephalogram (EEG) source localization method based on minimum norm estimation-generative adversarial network (MNE-GAN), comprising the following steps:
(1) generating virtual EEG source data in a motor state according to the Brodmann areas, computing an EEG forward propagation process based on a realistic head model to obtain a lead field matrix, mapping the virtual EEG source data to scalp electrode positions, and introducing electromyographic (EMG), electrocardiogramd electrooculographic (EOG) noises after the mapping process to generate EEG signals that simulate a real acquisition environment;
(2) preprocessing the EEG signals, comprising re-referencing, band-pass filtering, downsampling, artifact removal, and epoching, and extracting effective EEG features;
(3) constructing a generative adversarial network model based on minimum norm estimation (MNE-GAN model), wherein in the MNE-GAN model, a generator is configured to use the preprocessed virtual EEG data as a training set, learn spatial-temporal features of EEG signals through multi-layer convolution operations, and output high-resolution source-space averaged EEG signals in a block-wise manner according to the Brodmann areas, thereby establishing a mapping model from EEG signals to brain source distributions; and a discriminator is configured to use the source-space averaged EEG signals generated by the generator as fake samples and the EEG signals simulated in the step (1) as real samples for adversarial training, use binary cross-entropy loss as a loss function, compute a generator loss based on the discrimination results, and introduce a minimum norm inverse solution into the generator loss;
(4) training the MNE-GAN model using a staged training strategy: first, training the discriminator using noise signals, alternately training the generator and the discriminator until the model converges, and finally introducing the minimum norm inverse solution as a supervisory term for joint optimization; and
(5) preprocessing real EEG data according to the method as described in the step (2), inputting the preprocessed data into the trained MNE-GAN model, outputting signals corresponding to regions of the cerebral cortex, and performing localization according to intensities of the output signals.

2. The method according to claim 1, wherein the step (1) comprises:
selecting motor-related regions according to the Brodmann areas, assigning brain region labels, and dividing the motor-related regions into primary activation regions, synergistic regions, and background regions;
initializing source dipoles, simulating brain activity over different time periods in the selected activation regions using sinusoidal waves with different frequencies and amplitudes according to labels in different regions, simulating noise and low-frequency activity in the background regions using random noise and low-frequency activity, and assigning amplitudes to the source dipoles using the simulated signals; and
computing a forward solution of EEG signals in the motor-related regions according to a forward model, mapping the EEG signals in a source space onto the electrodes; and introducing ECG, EMG and EOG noises, as well as a random noise to simulate EEG signals acquired under realistic conditions.

3. The method according to claim 1, wherein the step (2) comprises:
performing band-pass filtering on the data in a range of 0.3 Hz-45 Hz, removing outliers, and repairing bad channels using a linear interpolation method;
removing EMG, ECG, and EOG interferences in the EEG signals using an independent component analysis (ICA) algorithm; and
segmenting the processed signals, and performing baseline correction and common average referencing.

4. The method according to claim 3, wherein the generator adopts a convolutional neural network (CNN) combined with fully connected layers to extract EEG signal features, comprising:
taking the preprocessed EEG signal X∈R^{c×t}, with c electrode channels and time length t, as input to feed into a convolutional layer for feature extraction, and performing dimensionality reduction through a pooling layer; and an input feature of a k^{th} layer is h^{k-1}, and a convolution operation of this layer is expressed as: h^{k}=ƒ(Conv(h^{k-1},ω^{k},b^{k})), wherein ƒ(⋅) denotes a ReLU activation function, w denotes weight parameters, and b denotes bias, and EEG encoded data s are finally obtained;
decoding the data s encoded by the convolutional layers using the fully connected layers, and the process is expressed as: h^{m}=ƒ(ω^{m}*h^{m-1}+b^{m}), and EEG features are finally obtained; and
performing block-wise mapping, selecting the motor-related regions according to the Brodmann areas, and mapping each block separately; when each block has r signal sources in the source space, a single source-averaged EEG signal A(x)∈R^{1*t} corresponding to an average signal of r signal sources in the source space is obtained by mapping, and all blocks are concatenated to output an overall source-averaged EEG signal G(x).

5. The method according to claim 1, wherein in a discriminator model, a signal input to the discriminator is recorded as S(x); and after passing through the convolutional layers and the fully connected layers, the signal is then output through a Sigmoid activation function to obtain a probability D(S(x)) that an input sample is a real sample;
a loss function of the discriminator is calculated using binary cross-entropy, a loss for the real sample is −log(D(R(x))), a loss for generated sample is −log(1−D(G(x))), and a final loss of the discriminator is an average of the two items:
$${Loss}_{Discriminator} = - \frac{1}{N} {\sum }_{i = 1}^{N} - log ( D ( R ( {x}_{i} ) ) ) - log ( 1 - D ( G ( {x}_{i} ) ) )$$
wherein N denotes a number of samples.

6. The method according to claim 5, wherein a loss of the generator is as follows: an adversarial loss is calculated based on the probability D(S(x)) output by the discriminator that an input sample is a real sample:
$${Loss}_{GAN} = - \frac{1}{N} {\sum }_{i = 1}^{N} - log ( D ( G ( {x}_{i} ) ) )$$
an initial loss function of the generator is only the adversarial loss; and after the generated results become stable, a minimum norm constraint loss function (MNE) is introduced:
$${Loss}_{mne} = \frac{1}{N} {\sum }_{i = 1}^{N} {‖ G ( {x}_{i} ) - {G}_{mne}^{i} ‖}_{2}^{2}$$
wherein G(xᵢ) denotes an output of the generator, and
$${G}_{mne}^{i}$$
denotes a minimum norm inverse solution corresponding to data xᵢ; an inverse solution MNE(x) is computed and obtained;
according to the Brodmann areas, source signals of the motor-related regions are selected to serve as final results, and regional signals are averaged to obtain a minimum norm inverse solution reference
$${G}_{mne}^{i} ;$$
and a final generator loss is obtained by weighting the adversarial loss and the minimum norm constraint loss:
$${Loss}_{Ge nerator} = {Loss}_{G A N} + {γ}_{mne} * {Loss}_{mne}$$
wherein γₘₙₑ denotes a weighting coefficient of the minimum norm loss and is used to balance the influence of various loss terms.

7. The method according to claim 6, wherein the step (5) comprises:
pretraining the model, fixing parameters of the generator, and iteratively updating parameters of the discriminator by using noise as fake samples and virtual source-averaged EEG signals as real samples;
performing adversarial training using the pretrained discriminator and the well-designed generator, and alternately iterating the parameters until convergence; and
after the model reaches preliminary convergence, introducing Lossₘₙₑ and γₘₙₑ for joint training until LOSS Generator decreases and converges.

8. A motor-related EEG source localization system based on MNE-GAN, comprising:
a virtual EEG source data generation module, configured to generate virtual EEG source data in a motor state according to Brodmann areas, compute an EEG forward propagation process based on a realistic head model to obtain a lead field matrix, map the virtual EEG source data to scalp electrode positions, and introduce electromyographic (EMG), electrocardiogram and electrooculographic (EOG) noises after the mapping process to generate EEG signals that simulate a real acquisition environment;
an EEG signal preprocessing module, configured to preprocess the EEG signals, comprising re-referencing, band-pass filtering, downsampling, artifact removal, and epoching, and extracting effective EEG features;
an MNE-GAN model construction module, configured to construct a generative adversarial network model based on minimum norm estimation (MNE-GAN model); in the MNE-GAN model, a generator is configured to use the preprocessed virtual EEG data as a training set, learn spatial-temporal features of EEG signals through multi-layer convolution operations, and output high-resolution source-space averaged EEG signals in a block-wise manner according to the Brodmann areas, thereby establishing a mapping model from EEG signals to brain source distributions; and
a discriminator is configured to use the source-space averaged EEG signals generated by the generator as fake samples and the EEG signals simulated by the virtual EEG source data generation module as real samples for adversarial training, use binary cross-entropy loss as a loss function, compute a generator loss based on the discrimination results, and introduce a minimum norm inverse solution into the generator loss;
a model training module, configured to train the MNE-GAN model using a staged training strategy: first, the discriminator is trained using noise signals, the generator and the discriminator are then alternately trained until the model converges, and finally the minimum norm inverse solution is introduced as a supervisory term for joint optimization; and
a source localization module, configured to preprocess real EEG data according to a method of the EEG signal preprocessing module, input the preprocessed data into the trained MNE-GAN model, output signals corresponding to regions of the cerebral cortex, and perform localization according to intensities of the output signals.

9. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 1 is implemented.

10. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 2 is implemented.

11. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 3 is implemented.

12. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 4 is implemented.

13. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 5 is implemented.

14. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 6 is implemented.

15. A computer device, comprising: one or more processors, a memory, and one or more programs, wherein the one or more programs are stored in the memory and are configured to be executed by the one or more processors, and when the programs are executed by the processors, the steps of the motor-related EEG source localization method based on MNE-GAN according to claim 7 is implemented.

16. A computer-readable storage medium having a computer program stored thereon, wherein when the computer program is executed by a processor, the steps of the motor-related EEG source localization method based on MNE-GAN according to claim 1 is implemented.

17. A computer-readable storage medium having a computer program stored thereon, wherein when the computer program is executed by a processor, the steps of the motor-related EEG source localization method based on MNE-GAN according to claim 2 is implemented.

18. A computer-readable storage medium having a computer program stored thereon, wherein when the computer program is executed by a processor, the steps of the motor-related EEG source localization method based on MNE-GAN according to claim 3 is implemented.

19. A computer-readable storage medium having a computer program stored thereon, wherein when the computer program is executed by a processor, the steps of the motor-related EEG source localization method based on MNE-GAN according to claim 4 is implemented.

20. A computer-readable storage medium having a computer program stored thereon, wherein when the computer program is executed by a processor, the steps of the motor-related EEG source localization method based on MNE-GAN according to claim 5 is implemented.
